(19) United States
(12) Patent Application Publication
(10) Pub. No.: US 2019/0125669 A1
(43) Pub. Date: May 2, 2019

(54) SYSTEMS AND METHODS FOR STEROIDAL GELS

(71) Applicant: Children's Medical Center Corporation, Boston, MA (US)

(72) Inventors: Daniel S. Kohane, Newton, MA (US); Qian Liu, Mountain View, CA (US)

(73) Assignee: Children's Medical Center Corporation, Boston, MA (US)

(21) Appl. No.: 16/094,125

(22) PCT Filed: Apr. 14, 2017
(86) PCT No.: PCT/US17/27520

§ 371 Date: Oct. 16, 2018

Related U.S. Application Data
(60) Provisional application No. 62/323,712, filed on Apr. 17, 2016.

(51) Int. Cl. A61K 9/06; A61K 31/661; A61K 31/445; A61K 9/00

(52) U.S. Cl. CPC A61K 9/06; A61K 31/661; A61K 33/06; A61K 9/0019; A61K 31/445

(57) ABSTRACT

The present invention generally relates to compositions, such as gels comprising steroids. In certain aspects, the steroids are modified such that they can form gels, e.g., when complexed to metal ions. In some cases, the steroids form fibers, such as nanofibers, within the gel. For instance, in one set of embodiments, a steroid may be relatively hydrophobic, and/or modified to include a phosphate moiety that is able to complex to metal ions to form the gel. In some cases, such gels may be administered to a subject, e.g., through injection. Other aspects of the invention are generally directed to methods of making or using such gels, kits comprising such gels, or the like.

### RELATED APPLICATIONS

This application claims the benefit of U.S. Provisional Patent Application Serial No. 62/323,712, filed Apr. 17, 2016, entitled “Systems and Methods for Steroidal Gels,” by Kohane, et al., incorporated herein by reference in its entirety.

### FIELD

The present invention generally relates to gels comprising steroids.

### BACKGROUND

Drug delivery systems generally entail a carrier to deliver therapeutic agents. This has been true of a wide range of particulate formulations and hydrogels. The biomaterials of which drug delivery systems are composed may require complex syntheses, and are sometimes expensive. They may have problems relating to biocompatibility. The residual debris of drug delivery systems may remain within tissues far beyond the duration of therapeutic effect of the delivered drug. There are also frequently limitations to the physical or chemical loading of drugs within the delivery systems, i.e., a substantial proportion is carrier, not drug. Consequently, drug delivery systems where there is no or minimal carrier are potentially attractive.

### SUMMARY

The present invention generally relates to gels comprising steroids. The subject matter of the present invention involves, in some cases, interrelated products, alternative solutions to a particular problem, and/or a plurality of different uses of one or more systems and/or articles.

In one aspect, the present invention is directed to a composition. In some cases, the composition may be a gel or a fiber, such as a nanofiber.

In accordance with one set of embodiments, the composition comprises a steroid comprising a phosphate moiety and a core portion, the core portion having a logP value greater than or equal to 1, complexed via Ca²⁺ and/or Ba²⁺ metal ions to form a hydrogel. The steroid may in some embodiments form at least 50% by dry weight of the hydrogel.

The composition, in another set of embodiments, includes a steroid comprising a phosphate moiety and a core portion, the core portion having no formal charged moieties at a pH of 7, the steroid complexed via Ca²⁺ and/or Ba²⁺ metal ions to form a nanofiber.

In yet another set of embodiments, the composition comprises a hydrogel comprising a steroid and Ca²⁺ and/or Ba²⁺ metal ions, the steroid comprising a phosphate moiety and being present within the hydrogel at a concentration of at least 8 mM, and the metal ions being present within the hydrogel at a concentration of at least 8 mM.

According to still another set of embodiments, the composition includes a nanofiber formed from a complex of (a) a steroid comprising a phosphate moiety, and (b) Ca²⁺ and/or Ba²⁺ metal ions, the steroid and the metal ions present within the nanofiber at a molar ratio of between about 2:1 and about 1:2.

In another aspect, the present invention is generally directed to a method of making a hydrogel. In one set of embodiments, the method includes an act of exposing a steroid comprising a phosphate moiety and a core portion to metal ions such as Ca²⁺ and/or Ba²⁺ metal ions to form a hydrogel. In some cases, the core portion may have a logP value greater than or equal to 1. In certain instances, the core portion may have no formal charged moieties at a pH of 7.

In another aspect, the present invention encompasses methods of making one or more of the embodiments described herein, for example, a gel formed from a steroid. In still another aspect, the present invention encompasses methods of using one or more of the embodiments described herein, for example, a gel formed from a steroid.

Other advantages and novel features of the present invention will become apparent from the following detailed description of various non-limiting embodiments of the invention when considered in conjunction with the accompanying figures.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1F illustrate various steroids and hydrogels in one set of embodiments of the invention;

FIGS. 2A-2B illustrate the characterization of a DexP-hydrogel in accordance with certain embodiments of the invention;

FIGS. 3A-3C illustrate in vitro drug release from and degradation of a DexP-hydrogel in another embodiment of the invention;

FIG. 4 illustrates plasma levels of dexamethasone after subcutaneous injection in accordance with another embodiment of the invention;

FIG. 5 illustrates durations of motor and sensory blockades, in accordance with certain embodiments of the invention;

FIG. 6 illustrates injection of certain materials according to another embodiment of the invention;

FIG. 7 illustrates representative IR spectra of certain hydrogels prepared according to yet another embodiment of the invention;

FIGS. 8A-8B illustrate a DexP hydrogel in accordance with yet another embodiment of the invention;

FIGS. 9A-9B illustrate self-assembled structures of certain embodiments of the invention;

FIGS. 10A-10B illustrate rheological characterization a hydrogel in ano embodiment of the invention:

FIGS. 11A-11B illustrate rheological characterization of another hydrogel n yet another embodiment of the invention:

FIG. 12 illustrates viability of certain cells exposed to a hydrogel in accordance in still another embodiment of the invention; and

FIGS. 13A-13D illustrates characterization of yet another embodiment of the invention.

### DETAILED DESCRIPTION

The present invention generally relates to compositions, such as gels comprising steroids. In certain aspects, the steroids are modified such that they can form gels, e.g., when complexed to metal ions. In some cases, the steroids form fibers, such as nanofibers, within the gel. For instance, in one set of embodiments, a steroid may be relatively hydrophobic, and/or modified to include a phosphate moiety that is able to complex to metal ions to form the gel. In some cases, such gels may be administered to a subject, e.g., through injection. Other aspects of the invention are generally directed to methods of making or using such gels, kits comprising such gels, or the like.

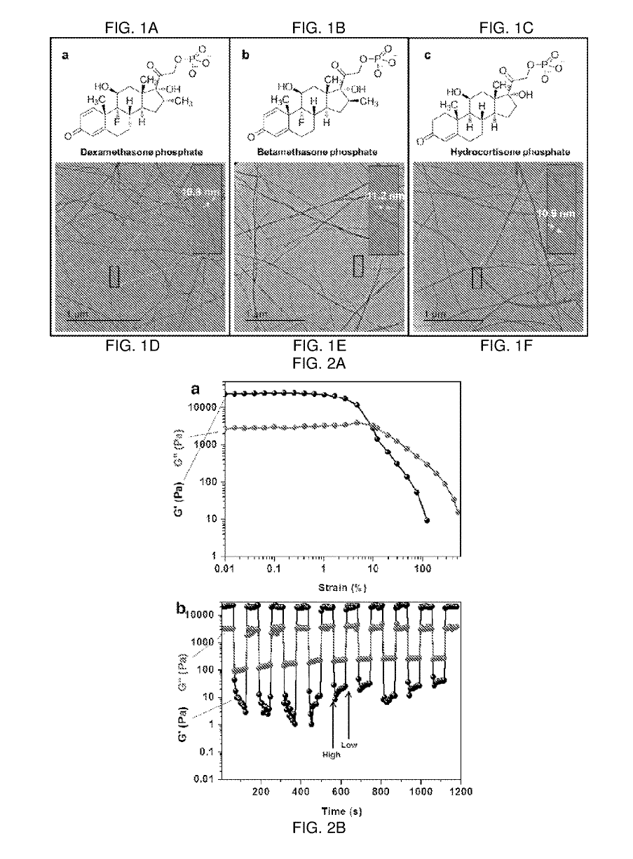

In one aspect, the present invention is generally directed to a composition comprising a gel formed from a steroid. A gel typically contains fibers or strands that form a network. See, e.g., FIGS. 1D-1F. Interstices within the network may allow water or another liquid to penetrate, generally giving a gel less rigid material properties, e.g., as compared to crystalline materials. It should be understood that, as described herein, the steroid typically forms an integral part of the gel, rather than merely being contained within the gel (e.g., dissolved or suspended within water contained within the gel). Thus, for example, the steroid may be formed into fibers or strands, such as nanofibers, that form at least part of the structure of the gel. However, in some embodiments, a gel may additionally contain other species contained therein, for instance, water, or other drugs, ions, etc., and/or there may be other materials in addition to steroids that form the structure of the gel, e.g., formed into fibers or strands that form the gel. A gel containing water is often referred to as a hydrogel.

In one set of embodiments, the steroids may form a significant portion of the structure of the gel. For instance, at least about 30% of the structure of the gel (e.g., including fibers such as nanofibers) may comprise a steroid, i.e., ignoring water or other materials contained within the gel but do not form part of the gel structure (e.g., the fibers or strands within the gel), or in other words, the dry weight of the gel in the absence of water. In some cases, at least about 40%, at least about 50%, at least about 60%, at least about 70%, at least about 75%, at least about 80%, at least about 85%, at least about 90%, or at least about 95% of the structure of the gel, or the dry weight of the gel, may comprise a steroid.

In certain embodiments, the steroid may be stabilized to form fibers or other structures via one or more charged ions. For example, the steroid may contain a phosphate moiety, which may complex with metal ions to stabilize the structure. See, e.g., FIG. 9B. The metal ions may be, for instance, alkaline earth metal ions such as Ca²⁺ or Ba²⁺, which may interact with the negatively changed phosphate moiety in order to stabilize the steroid. The steroid may also be relatively hydrophobic in some cases. For instance, portions of the steroid other than the phosphate moiety may be relatively hydrophobic. Such hydrophobicity may assist in stability or self-assembly of the steroid into fibers or other structures that form the gel.

A steroid generally comprises 4 fused rings (three 6-carbon rings and one 5-carbon ring) arranged in a certain configuration, e.g., as shown here, with the rings labeled A, B, C, and D as follows:

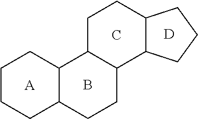

This structure (i.e., when the substituents are all H's) is often referred to as a gonane structure. However, in many steroids, one or more of these positions will be substituted. These positions are typically numbered as follows:

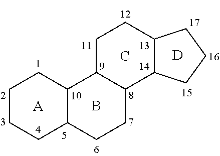

Thus, for example, many steroids will have a methyl group at the 10 position, a methyl group at the 13 position, and/or a carboxylate moiety at the 17 position. In some cases, there may also be an oxo moiety at the 3 position, and/or a hydroxyl moiety at the 17 position. Other examples of substituents that may appear on a steroid include halogens (e.g., —F, —Cl, —Br, —I, etc.), alkyls (e.g., methyl, ethyl, etc.), or hydroxyls. In addition, in some cases, some of the carbon atoms within the structure may be connected by double bonds, e.g., the 1 and 2 carbons and/or the 4 and 5 carbons. Specific non-limiting examples of steroids include corticosteroids such as glucocorticoids. Many steroids are readily available commercially.

In one set of embodiments, the carboxylate moiety at the 17 position may be an acetyl moiety (—C(O)—CH₃), and in some cases, the phosphate moiety may be bonded to the core of the steroid through the methyl group of the acetyl moiety (i.e., to form a structure —C(O)—CH₂—OPO₃²⁻). Many steroids having such phosphate moieties can be commercially obtained, or a steroid can be reacted to include such a phosphate moiety, for instance, by reaction with o-phosphoric acid, pyrophosphoryl tetrachloride, or other suitable phosphorylating reagents. In some embodiments, however, the phosphate moiety (OPO₃²⁻) may be directly bonded to the 17 position (i.e., without necessarily including a carboxylate moiety).

The core portion of the steroid, other than the phosphate moiety, may be relatively hydrophobic in some cases. In some cases, the phosphate moiety may be the only charged portion of the steroid, i.e., the core portion of the steroid may have no formal charged moieties at a pH of 7. For instance, the core portion of the steroid may be free of carboxylic acid groups (COO—) or other such charged moieties. In certain embodiments, the core portion of the steroid has a logP value greater than or equal to 1, greater than or equal to 1.2, greater than or equal to 1.4, greater than or equal to 2, etc. where the logP value is determined in the absence of the phosphate moiety on the steroid. The logP value is typically determined in an octanol-water partitioning system under standard conditions (25° C. and 1 atm). In some cases, the steroid may comprise at least one hydroxyl group, and in some cases, each face of the steroid (e.g., the two faces formed on either side of the A-B-C-D rings) may comprise an equal number of hydroxyl groups. In some cases, without wishing to be bound by any theory, it is believed that such hydroxyl groups may participate in certain hydrogen bond interactions, which may also facilitate stabilization.

Specific non-limiting examples of steroids (including core portions and phosphate moieties include the following):

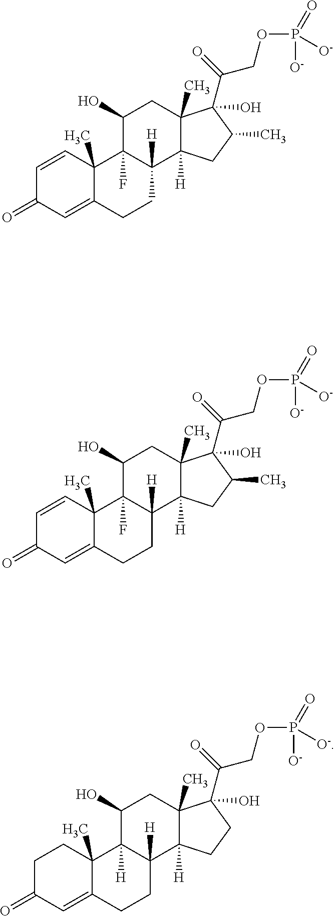

As previously discussed, the steroid may be associated with one or more ions to form fibers or strands that form the gel. For instance, the steroid may contain a negatively charged phosphate moiety, which may complex with one or more positively charged ions. Examples of positively charged ions include alkali metals or alkaline earth metals, such as Ca²⁺ and/or Ba²⁺, which may come from sources such as aqueous solutions of CaCl₂, CaSO₄, or BaCl₂. In some cases, at least about 30 mol % of the metal ions within the gel are alkaline earth metals, such as Ca²⁺ and/or Ba²⁺, and in some cases, at least about 40 mol %, at least about 50 mol %, at least about 60 mol %, at least about 70 mol %, at least about 75 mol %, at least about 80 mol %, at least about 85 mol %, at least about 90 mol %, or at least about 95 mol % of the metal ions within the gel are alkaline earth metals, such as Ca²⁺ and/or Ba²⁺.

In some cases, the steroid and the metal ion may be present within the gel, e.g., within fibers or strands that form the gel, at any suitable molar ratio, for instance, at a molar ratio of between 5:1 and 1:5, between 4:1 and 1:4, between 3:1 and 1:3, between 2:1 and 1:2, or between 1.5:1 and 1:1.5. Without wishing to be bound by any theory, it is believed that the complex occurs in a generally 1:1 molar ratio. However, excesses of either species that are present are also possible. In some cases, ratios other than 1:1 may result in some species not participating in complexation, although such ratios can still nevertheless form gels, and are accordingly contemplated in various embodiments of the invention.

In addition, in some cases, a certain concentration of steroid and/or metal ion may be present within the gel, e.g., within fibers or strands that form the gel. For instance, the concentration of steroid and/or metal ion may each independently be at least about 1 mM, at least about 2 mM, at least about 2.5 mM, at least about 3 mM, at least about 4 mM, at least about 5 mM, at least about 6 mM, at least about 7 mM, at least about 8 mM, at least about 10 mM, at least about 11 mM, at least about 12 mM, at least about 15 mM, etc. In some cases, the concentration of steroid and/or metal ion may each independently be at most 25 mM, at most 20 mM, or at most 15 mM. Combinations of any of these concentrations are also possible in certain embodiments.

In some cases, the steroids may complex to metal ions, such as Ca²⁺ and/or Ba²⁺, to form fibers or strands that form a network forming the gel. Non-limiting examples may be seen in FIGS. 1D-1F. In some cases, the fibers include nanofibers, i.e., fibers having an average cross-sectional diameter of less than 1 micrometer, e.g., typically measured in nanometers. For instance, the nanofiber may have an average cross-sectional diameter of less than about 1000 nm, less than about 300 nm, less than about 100 nm, less than about 50 nm, less than about 25 nm, or less than about 15 nm. In some cases, the nanofiber has an average cross-sectional diameter between 5 nm and 25 nm, between 5 nm and 15 nm, between 10 nm and 15 nm, etc. In some cases, the nanofibers may be loosely arranged and water may be present between the nanofiber, e.g., forming a hydrogel.

In some cases, the composition may exhibit properties such as self-healing, shear-thinning, or thixotropic properties. For example, the composition may include a gel that can exhibit decreased viscosity when subjected to shear strain. Without wishing to be bound any theory, it is believed that such properties arise due to the complexation between the steroid and the metal ions, which can break and re-form as necessary when stresses are applied. Thus, after stress has been applied, the complexes can re-form, thereby resulting in self-healing, shear-thinning, thixotropic, or other similar properties.

Other components may be present within the gel. For example, the gel may contain water and/or other suitable liquids, e.g., contained within the fibers, etc. forming the gel. In some cases, the water may contain other components, such as nutrients, salts, vitamins, hormones, drugs, or the like. For example, in one embodiment, the gel may contain an anesthetic, such as bupivacaine.

In some aspects, a complex of steroids and metal ions may form spontaneously, for example upon exposure of a steroid to metal ions such as Ca²⁺ and/or Ba²⁺ in solution. Thus, for example, a steroid and a solution containing metal ions (e.g., a dissolved calcium salt, a dissolved barium salt, etc.) may be mixed together to form a suitable gel. In some cases, this may occur under ambient conditions (e.g., 25° C. and 1 atm). Other materials, such as nutrients, salts, vitamins, hormones, drugs, or the like, may also present, e.g., during formation of the gel, and/or may be added afterwards, for example, through diffusion into the gel.

In some cases, such gels may be applied to a subject, such a human subject. For instance, in one set of embodiments, the gel may be contained within a suitable needle or a syringe for injection into a subject, or the gel may be implanted within a subject. Thus, another aspect provides a method of administering any composition of the present invention to a subject. When administered, the compositions of the invention are applied in a therapeutically effective, pharmaceutically acceptable amount as a pharmaceutically acceptable formulation.

As used herein, the term “pharmaceutically acceptable” is given its ordinary meaning. Pharmaceutically acceptable compositions are generally compatible with other materials of the formulation and are not generally deleterious to the subject. Any of the compositions of the present invention may be administered to the subject in a therapeutically effective dose. A “therapeutically effective” or an “effective” as used herein means that amount necessary to delay the onset of, inhibit the progression of, halt altogether the onset or progression of, diagnose a particular condition being treated, or otherwise achieve a medically desirable result. The terms “treat,” “treated,” “treating,” and the like, generally refer to administration of the inventive compositions to a subject. When administered to a subject, effective amounts will depend on the particular condition being treated and the desired outcome. A therapeutically effective dose may be determined by those of ordinary skill in the art, for instance, employing factors such as those further described below and using no more than routine experimentation.

In administering the compositions of the invention to a subject, dosing amounts, dosing schedules, routes of administration, and the like may be selected so as to affect known activities of these compositions. Dosages may be estimated based on the results of experimental models, optionally in combination with the results of assays of compositions of the present invention. Dosage may be adjusted appropriately to achieve desired drug levels, local or systemic, depending upon the mode of administration. The doses may be given in one or several administrations per day.

The dose of the composition to the subject may be such that a therapeutically effective amount of the composition reaches the active site of the composition within the subject. The dosage may be given in some cases at the maximum amount while avoiding or minimizing any potentially detrimental side effects within the subject. The dosage of the composition that is actually administered is dependent upon factors such as the final concentration desired at the active site, the method of administration to the subject, the efficacy of the composition, the longevity of the composition within the subject, the timing of administration, the effect of concurrent treatments (e.g., as in a cocktail), etc. The dose delivered may also depend on conditions associated with the subject, and can vary from subject to subject in some cases. For example, the age, sex, weight, size, environment, physical conditions, or current state of health of the subject may also influence the dose required and/or the concentration of the composition at the active site. Variations in dosing may occur between different individuals or even within the same individual on different days. It may be preferred that a maximum dose be used, that is, the highest safe dose according to sound medical judgment. Preferably, the dosage form is such that it does not substantially deleteriously affect the subject.

Administration of a composition of the invention may be accomplished by any medically acceptable method which allows the composition to reach its target. The particular mode selected will depend of course, upon factors such as those previously described, for example, the particular composition, the severity of the state of the subject being treated, the dosage required for therapeutic efficacy, etc. As used herein, a “medically acceptable” mode of treatment is a mode able to produce effective levels of the composition within the subject without causing clinically unacceptable adverse effects.

Any medically acceptable method may be used to administer the composition to the subject. The administration may be localized (i.e., to a particular region, physiological system, tissue, organ, or cell type) or systemic, depending on the condition to be treated. The composition may be administered via injection in some cases. The composition also may be administered orally, vaginally, rectally, buccally, pulmonary, topically, nasally, transdermally, through parenteral injection or implantation, via surgical administration, or any other method of administration where access to the target by the composition of the invention is achieved. Examples of parenteral modalities that can be used with the invention include intravenous, intradermal, subcutaneous, intracavity, intramuscular, intraperitoneal, epidural, or intrathecal. Examples of implantation modalities include any implantable or injectable drug delivery system.

In certain embodiments of the invention, the administration of the composition of the invention may be designed so as to result in sequential exposures to the composition over a certain time period, for example, hours, days, weeks, months or years. This may be accomplished, for example, by repeated administrations of a composition of the invention by one of the methods described above, or by a sustained or controlled release delivery system in which the composition is delivered over a prolonged period without repeated administrations.

Use of a long-term release implant may be particularly suitable in some embodiments of the invention. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

Administration of the composition can be alone, or in combination with other therapeutic agents and/or compositions. In some embodiments, the compositions of the invention include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers that may be used with the active compound. Examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. Suitable concentrations can be determined by those of ordinary skill in the art, using no more than routine experimentation. Those of ordinary skill in the art will know of other suitable formulation ingredients, or will be able to ascertain such, using only routine experimentation.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. Those of skill in the art can readily determine the various parameters for preparing and formulating the compositions of the invention without resort to undue experimentation.

The present invention also provides any of the above-mentioned compositions in kits, optionally including instructions for use of the composition. Instructions also may be provided for administering the composition by any suitable technique as previously described, for example, via injection or another known route of drug delivery. The kits described herein may also contain one or more containers, which may contain the inventive composition and other ingredients as previously described. The kits also may contain instructions for mixing, diluting, and/or administrating the compositions of the invention in some cases. The kits also can include other containers with one or more solvents, surfactants, preservative and/or diluents (e.g., normal saline (0.9% NaCl), or 5% dextrose) as well as containers for mixing, diluting or administering the components in a sample or to a subject in need of such treatment.

U.S. Provisional Patent Application Ser. No. 62/323,712, filed Apr. 17, 2016, entitled “Systems and Methods for Steroidal Gels,” by Kohane, et al., is incorporated herein by reference in its entirety.

The following examples are intended to illustrate certain embodiments of the present invention, but do not exemplify the full scope of the invention.

### EXAMPLE 1

Drug-based hydrogels produced by a rapid, simple, and efficient strategy would be desirable. Ideally, to facilitate minimally invasive application in vivo by direct injection, such supramolecular hydrogels should exhibit decreased viscosity under shear stress (shear-thinning) and rapid recovery when the applied stress is relaxed (self-healing).

This example presents shear-thinning and self-healing hydrogels based on steroid drugs (anti-inflammatory glucocorticoid receptor agonists) such as dexamethasone, betamethasone, and hydrocortisone, that can be formed in seconds by simple coordination reactions. In clinical practice, glucocorticoid receptor agonists are widely used as anti-inflammatory agents to treat arthritis, asthma, and many other conditions. They can have numerous other effects, for example on glucose metabolism and blood pressure. The indications and side effects of such drugs are well established. These steroid drugs have a rigid hydrophobic core composed of four fused rings and several hydroxyl groups which can be used to form hydrogen bonds (FIG. 1).

The hydrogel design used in this example was based on self-assembly between the steroid nuclei of phosphate salts of steroid drugs, and coordination interactions between their phosphate groups and alkaline earths metal ions.

Dexamethasone phosphate (DexP), betamethasone phosphate (BetP), or hydrocortisone phosphate (HydP) were combined with the metal ions Mg²⁺, Ca²⁺, or Ba²⁺, as shown in Table 1. The combination of 25 mM Ca²⁺ and 25 mM steroid drugs (DexP, BetP, or HydP) reliably formed gels (termed DexP-hydrogel, BetP-hydrogel, and HydP-hydrogel, respectively) with an extended nanofiber network on TEM (FIG. 1). The diameter of nanofibers was approximately 11 nm in all of the tested hydrogels. The similarity in nanofiber diameter may be due to the similarity in the molecular structure of these drugs. These hydrogels maintained their integrity and shape (e.g. remaining in position in an inverted tube) for at least one month at room temperature. Reactions with Ba²⁺ produced precipitates which became hydrogels when left at room temperature overnight. The different outcomes between groups may be due to differences in coordinating ability, coordination number, and solubility products between the various steroid drugs and alkaline-earth metal ion pairs.

FIG. 1 shows characterization of the hydrogels with TEM. FIGS. 1A-1C: chemical structure of the steroid drugs used in this example. FIGS. 1D-1F: TEM images of their associated hydrogels. The insets show the HR-TEM images. The concentrations of the steroid drugs and Ca²⁺ were both 25 mM. The scale bar is 1 micrometer.

TABLE 1
Gelation of steroid drugs and alkaline-earth metal ion.

| | Ca²⁺ concentration | | | | Ba²⁺ concentration | | | |
|---|---|---|---|---|---|---|---|---|
| | 2.5 | 6.25 | 12.5 | 25 | 2.5 | 6.25 | 12.5 | 25 |
| Dexamethasone | L/H | L/H | H | H | L | L | P−>H | P−>H |
| phosphate | | | | | | | | |
| Betamethasone | L/H | L/H | H | H | L | L | P−>H | P−>H |
| phosphate | | | | | | | | |
| Hydrocortisone | L/H | L/H | H | H | L | L | P−>H | P−>H |
| phosphate | | | | | | | | |

The molar ratio of the steroid drug to the alkaline earth metal ion was 1:1 for all of the above samples. L indicates that a liquid was formed; P indicates that a precipitate was formed; H indicates that a hydrogel was formed; L/H indicates that a mixture of a hydrogel and a liquid was formed; P->H indicates that a precipitate formed initially which converted to a hydrogel overnight.

### EXAMPLE 2

Subsequent experiments focused on the hydrogel formed by DexP and Ca²⁺ because it formed promptly. If the molar ratio of DexP to Ca²⁺ in the initial mixture was changed from 1:1 to 2:1 or 2:3, a hydrogel was still obtained. TEM images showed similar nanofiber diameters in gels with DexP:Ca²⁺=1:1, 2:1, and 2:3. Interestingly, the molar ratio of DexP to Ca²⁺ in hydrogel nanofibers (after hydrogel formation) was ˜1:1 by inductively coupled plasma mass spectrometry (ICP-MS) and high-performance liquid chromatography (HPLC), suggesting that deviations from a 1:1 molar ratio resulted in free DexP or Ca²⁺. The critical gelation concentration, which was defined as the lowest concentration of the DexP which leads to a stable gel, was ˜10 mM (DexP:Ca²⁺=1:1) as reflected by the partial gelation at concentration <10 mM. The hydrogel (DexP:Ca²⁺=1:1) did not undergo a gel-sol transition at a temperature as high as the boiling point of water.

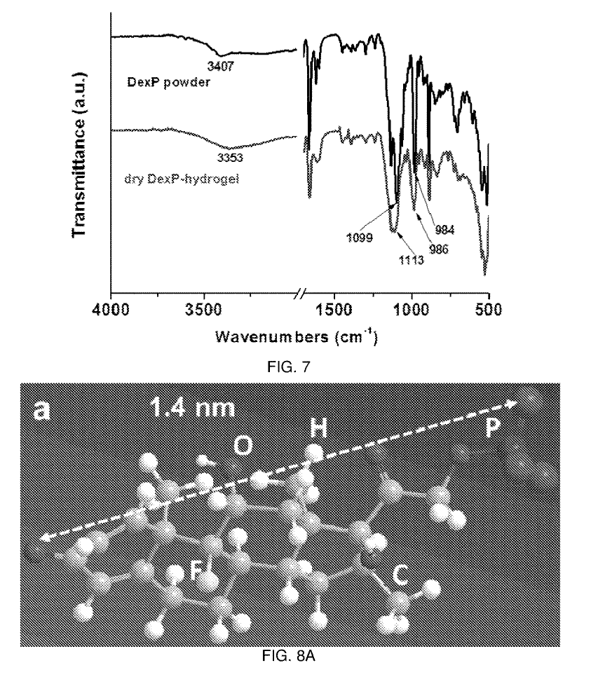

The assembly of the DexP-hydrogel was investigated by Fourier transform infrared (FTIR) spectroscopy and high-resolution TEM (HR-TEM). The FTIR bands in DexP powder (FIG. 7) at 1099 and 984 cm⁻¹, which were attributable to the characteristic antisymmetric and symmetric stretching vibrations of the phosphate group, were shifted to 1113 and 986 cm⁻¹ upon addition of Ca²⁺ and the consequent formation of a hydrogel, indicating that the phosphate group was involved in the coordination bonds. Also, after the formation of hydrogel, the O—H vibration band at 3407 cm⁻¹ in DexP was shifted to 3353 cm ⁻¹, indicating that the hydrogen bonds between DexP molecules was stronger in the hydrogel than in the powder. These results suggest that coordination interactions and hydrogen bonding play roles in gelation. Hydrophobic interaction could also play an important role in the self-assembly process, given the presence of the hydrophobic steroid nucleus in DexP.

FIG. 7 shows representative IR spectra of DexP powder and freeze-dried DexP-hydrogel. The concentration of DexP was 25 mM in the DexP-hydrogel, and the molar ratio of DexP to Ca²⁺ was 1:1.

### EXAMPLE 3

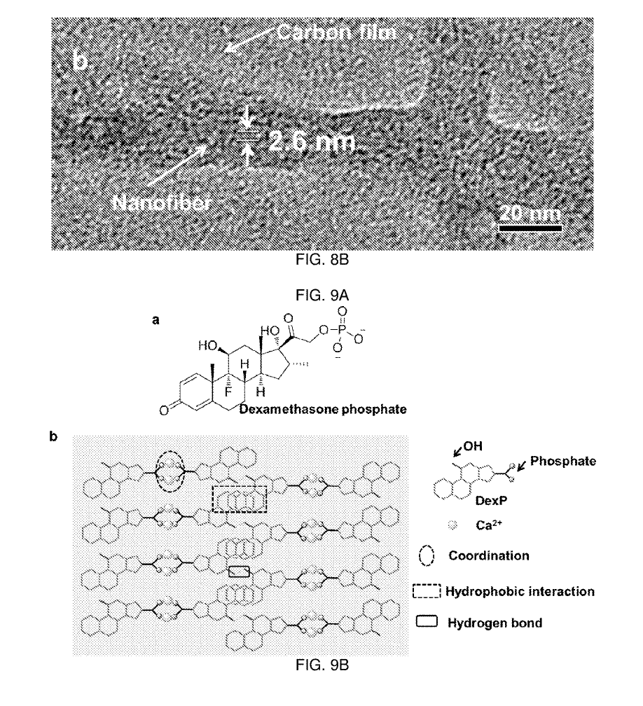

HR-TEM was performed to further define the structure of the hydrogel. FIG. 8 shows a hydrogel nanofiber surrounded by a carbon film. The hydrogel structure showed rows of dark material representing metal ions. The periodicity of the dark rows is approximately 2.6 nm, which is close to twice the length of the longest axis of DexP, 1.4 nm (calculated by ChemDraw).

FIG. 8A shows a 3D ball-and-stick model of DexP, while FIG. 8B shows HR-TEM images of the DexP-hydrogel. The concentration of DexP was 25 mM, and the molar ratio of DexP to Ca²⁺ was 1:1.

The FTIR and HR-TEM data and the fact that the DexP:Ca²⁺ ratio in nanofibers was 1:1 suggested a possible scheme of molecular packing (FIG. 9). Two DexP molecules would coordinate with two Ca²⁺ by their phosphate groups. The other end of the DexP molecules would interact with other DexP molecules by hydrophobic interactions and hydrogen bonds.

FIG. 9 shows self-assembly structures of the hydrogel. FIG. 9A shows a chemical structure of DexP-hydrogel. FIG. 9B shows a possible schematic of DexP-hydrogel nanofiber self-assembly.

### EXAMPLE 4

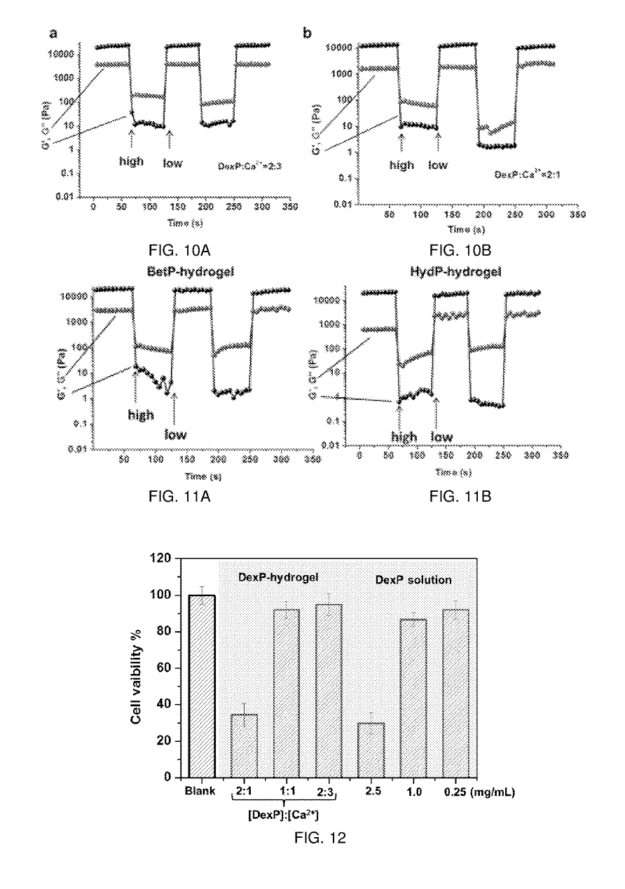

In this example, the mechanical properties of the DexP-hydrogel ([DexP]=25 mM, [Ca²⁺]=25 mM) were tested by performing oscillatory shear rheology (FIG. 2) which showed a strain (ε, epsilon) at yield of 9.8%. This is the strain at the cross point of G′ and G″, indicating the transition of the gel network to a liquid state; solution behavior: G′<G″, solid behavior: G′>G″.) The material properties of the DexP-hydrogel recovered completely and rapidly when transitioning from high magnitude strain (ε=50%) to low magnitude strain (ε=0.1%) (FIG. 2B). Recovery was rapid and complete over nine cycles of breaking and reforming, demonstrating the robust reversibility of the hydrogel mechanical properties. DexP-hydrogels with DexP:Ca²⁺=2:1 and 2:3 also exhibited fast recovery (FIG. 10)). Hydrogels made from BetP and Ca²⁺ ([BetP]=25 mM, [Ca²⁺]=25 mM) and HydP and Ca2+ ([HydP]=25 mM, [Ca²⁺]=25 mM) showed similar mechanical, shear-thinning, and self-healing properties (FIG. 11) to those of the DexP-hydrogel.

FIG. 2 shows rheological characterization of the DexP-hydrogel. FIG. 2A shows strain-dependent oscillatory shear rheology of the DexP-hydrogel. FIG. 2B shows step-strain measurements of the DexP-hydrogel over nine cycles, showing mechanical properties at low strain (0.1%) and high strain (50%). In both panels, the concentration of DexP was 25 mM, the molar ratio of DexP to Ca²⁺ was 1:1. Data are representative graphs of three experiments. G′ is the storage modulus, and G″ is the loss modulus. Arrows indicate the onset of high or low magnitude strains.

FIG. 10 shows rheological characterization of DexP-hydrogels with DexP:Ca²⁺ of (FIG. 10A) 2:3 and (FIG. 10B) 2:1. Graphs are representative step-strain measurements of three experiments under low magnitude strain (0.1%) and high magnitude strain (50%). The concentration of DexP was 25 mM in both hydrogels. G′ is the storage modulus and G″ is the loss modulus. Arrows indicate the onset of high or low magnitude strains.

FIG. 11 shows rheological characterization of the BetP- and HydP-hydrogels. Graphs are representative step-strain measurements of three experiments under low magnitude strain (0.1%) and high magnitude strain (50%). The concentrations of BetP and HydP were 25 mM, the molar ratio between steroid drug and Ca²⁺ was 1:1. G′ is the storage modulus and G″ is the loss modulus. Arrows indicate the onset of high or low magnitude strains.

### EXAMPLE 5

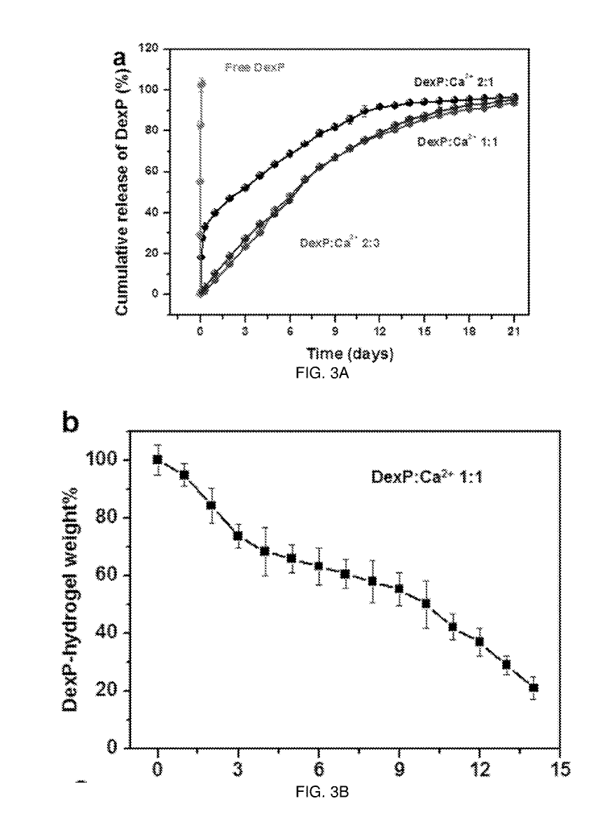

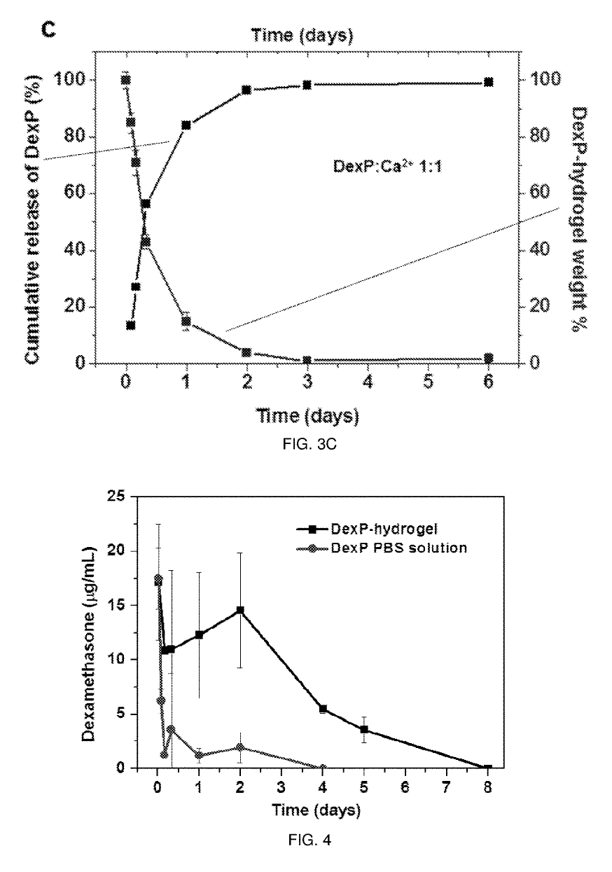

Hydrogels slowed the release of DexP from the upper chamber of a Transwell® system (see below) compared to the free drug in saline (FIG. 3). The kinetics of DexP release depended on the ratio of DexP to Ca²⁺. When the concentration of DexP was higher than that of Ca²⁺ ([DexP]:[Ca²⁺]=2:1), there was an initial burst release, suggesting free DexP in the hydrogel, and release plateaued after ˜2 weeks. Increasing the proportion of Ca²⁺ ([DexP]:[Ca²⁺]=1:1 or 2:3) removed burst release, and release occurred for ˜20 days. These results were consistent with the observation that the molar ratio of DexP and Ca²⁺ in the nanofibers of all DexP hydrogels was ˜1:1; presumably excess DexP would be present in the hydrogel as free drug. The degradation of DexP-hydrogel (FIG. 3B) was consistent with the release kinetics (FIG. 3A): ˜80% DexP-hydrogel ([DexP]:[Ca²⁺]=1:1) degraded after 14 days, in which time ˜80% of drug was released. In phosphate buffered saline (PBS), release of DexP and DexP-hydrogel degradation were both more rapid, achieving completion in ˜2 days (FIG. 3C). The accelerated drug release and degradation may be explained by interaction between the phosphate in PBS (10 mM) and the Ca²⁺ in the hydrogels.

The cytotoxicity of DexP-hydrogels ([DexP]:[Ca²⁺]=2:1, 1:1, or 2:3), and free DexP were assessed by exposing C2C12 cells to them for 24 h in a Transwell® system where the gels were separated from the cells by a permeable membrane (FIG. 12) and measuring cell viability by MTS (3-(4,5-dimethylthiazol-2-yl)-5-(3-carboxymethoxyphenyl)-2-(4-sulfophenyl)-2H-tetrazolium) assay. There was a concentration-dependent reduction of cell viability with increasing concentration of free DexP. Cell viability was minimally reduced by the DexP-hydrogels with [DexP]:[Ca²⁺]=1:1 (p=0.16) and 2:3 (p=0.24). Significant cytotoxicity was seen with [DexP]:[Ca²⁺]=2:1(p<0.005 compared to untreated cells), which was likely due to the presence of high concentrations of free DexP. The concentration of free DexP in the cell culture media incubated with the DexP-hydrogel ([DexP]:[Ca²⁺]=2:1) for 24 h was 2.5 mg/mL (calculated from the release profile in FIG. 3), and the reduction of cell viability in that group was similar to that from 2.5 mg/mL of free DexP (p=0.31, p<0.005 compared to untreated cells). 25 mM DexP-hydrogels with the ratio [DexP]:[Ca²⁺]=1:1 were used in subsequent experiments.

FIG. 3 shows in vitro drug release from and degradation of DexP-hydrogels. FIG. 3A shows cumulative release of DexP from DexP-hydrogels with various molar ratios of DexP to Ca²⁺, and from free DexP solution in saline. FIG. 3B shows degradation of DexP-hydrogel in saline ([DexP]:[Ca²⁺]=1:1). FIG. 3C shows cumulative release of DexP from DexP-hydrogels and degradation of DexP-hydrogel in PBS, ([DexP]:[Ca²⁺]=1:1). The concentration of DexP was 25 mM in all groups. Data are means+/−SDs (n=4).

FIG. 12 shows viability of C2C12 cells exposed to DexP hydrogels ([DexP]=25 mM or 12.9 mg/mL) with varying ratios of DexP to Ca²⁺ ([DexP]:[Ca²⁺]=2:1, 1:1, and 2:3) and to free DexP solution for 24 hours. Blank=no free DexP, no DexP-hydrogel. Data are mean+/−SD (n=4).

### EXAMPLE 6

In this example, the pharmacokinetics of DexP in the bloodstream after subcutaneous administration of DexP-hydrogel ([DexP]:[Ca²⁺]=1:1) in rats were determined by HPLC. Plasma levels of dexamethasone remained elevated (>10 micrograms/mL) for 48 hours after injection of 600 microliters of DexP-hydrogel (FIG. 4; 25 mM DexP, [DexP]:[Ca²⁺]=1:1). (These doses and the resulting concentrations are much higher than would be employed clinically, but were used here for proof of concept). Drug in the blood was detected as both DexP and dexamethasone; dexamethasone phosphate is rapidly converted to dexamethasone in plasma. The dexamethasone level then declined gradually, and became lower than the detection limit of the HPLC method (˜0.5 micrograms/mL) at 192 hours (8 days). In the group treated with free 25 mM DexP in PBS solution, the plasma dexamethasone level decreased rapidly to 1.2 micrograms/mL at 4 hours post-injection.

The tissue dwell time of DexP-hydrogel was assessed by euthanizing animals at predetermined time points after injection and dissecting the injection site. Hydrogel was observed subcutaneously 5 min after injection. Only a very small amount of hydrogel remained 5 days after injection. At 8 days, no hydrogel residue could be seen. These results demonstrate that the hydrogels did not persist in vivo after drug release was complete.

In order to demonstrate the effectiveness of the DexP-hydrogel in vivo, it was loaded with the local anesthetic bupivacaine hydrochloride (BPV) in preparation for studying it in rat sciatic nerve blockade. That is an animal model which would allow assessment of the performance of DexP-hydrogel in two parameters: prolongation of the duration of sciatic nerve block and anti-inflammatory activity, since bupivacaine causes transient local inflammation.

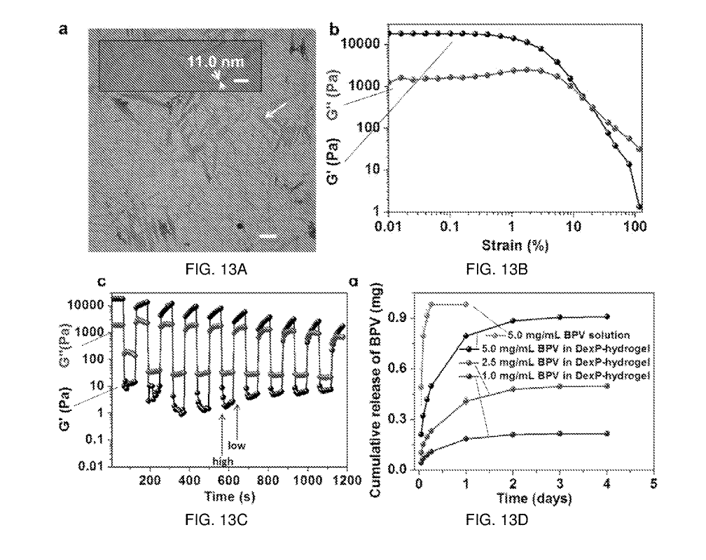

DexP-hydrogels were loaded with bupivacaine (BPV-DexP-hydrogel; FIG. 13; [DexP]=25 mM, [Ca²⁺]=25 mM, [BPV]=5 mg/mL (15.4 mM), [DexP]:[Ca²⁺]: [BPV]=1.62:1.62:1). TEM showed a nanofiber morphology with diameter of 11.0 nm (FIG. 13A) similar to that without bupivacaine, but with some nano- to microparticulate debris. Mechanical studies of the BPV- DexP-hydrogel showed a strain at yield of ε=20.2% (FIG. 13B). Step-strain measurements showed diminishing recovery of G′ with repeated strain steps (FIG. 10C). In the first cycle, 76% of G′ was recovered and within 6 s after withdrawal of the high magnitude strain (ε=50%). In the ninth cycle, only ˜10% of G′ was recovered. The weaker self-healing behavior of the BPV-DexP-hydrogel may be a result of the presence of bupivacaine, which interrupt the hydrogel self-assembly weakening its mechanical properties.

The hydrogel significantly slowed the release of bupivacaine in vitro at 37° C. compared to free drug (e.g., P<0.001 at 24 h; FIG. 13D), lasting at least 2 days. Released amount of bupivacaine increased with increasing drug loading.

FIG. 4 shows plasma levels of total dexamethasone (dexamethasone and dexamethasone phosphate) after subcutaneous injection of DexP-hydrogel (600 microliters) or dexamethasone phosphate PBS solution (200 microliters) in rats. The concentration of DexP was 25 mM in both groups, [DexP]:[Ca²⁺]=1:1. Data are mean+/−SDs (n=4).

FIG. 13 shows characterization of the BPV-DexP- hydrogel. FIG. 13A shows TEM of the BPV-DexP- hydrogel, scale bar is 500 nm. Arrow indicates micro- to nanoparticulate debris in the BPV-DexP-hydrogel. Insert: HR-TEM, the scale bar is 100 nm, the arrows indicate the diameter of a nanofiber. FIG. 3B shows representative (of three experiments) strain-dependent oscillatory shear rheology of the BPV-DexP-hydrogel. FIG. 3C shows representative (of three experiments) step-strain measurements the BPV-DexP- hydrogel over nine cycles. Arrows indicate the onset of high (50%) or low (0.1%) magnitude strains. FIG. 3D shows cumulative release of various concentrations of bupivacaine from BPV-DexP-hydrogel, data are mean+/−SDs (n=4; SDs too small to see). The concentration of DexP was 25 mM, the molar ratio of DexP to Ca²⁺ was 1:1. The concentration of bupivacaine in FIGS. 13A-13C is 5 mg/mL. G′ is the storage modulus, and G″ is the loss modulus.

### EXAMPLE 7

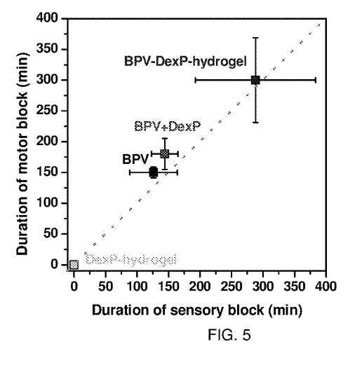

In this example, rats were injected at the sciatic nerve with 0.3 mL of test materials (n=6 in all groups) following which animals underwent neurobehavioral testing. DexP-hydrogel alone did not cause any impairment in sensory or motor function (FIG. 5). Sensory blockade from free 5 mg/mL bupivacaine solution lasted 126+/−38 min, and was not changed by co-injection with free DexP solution ([DexP]=25 mM, [BPV]=5 mg/mL (15.4 mM), 144+/−21 min, p=0.18). Sensory block from BPV-DexP-hydrogel (5 mg/mL (15.4 mM) bupivacaine) lasted 288+/−95 min, more than twice longer than that from bupivacaine alone (p=0.02). Motor blockade from BPV-DexP-hydrogel was generally similar to the duration of sensory blockade, as evidenced by their proximity to the line of unity in FIG. 5.

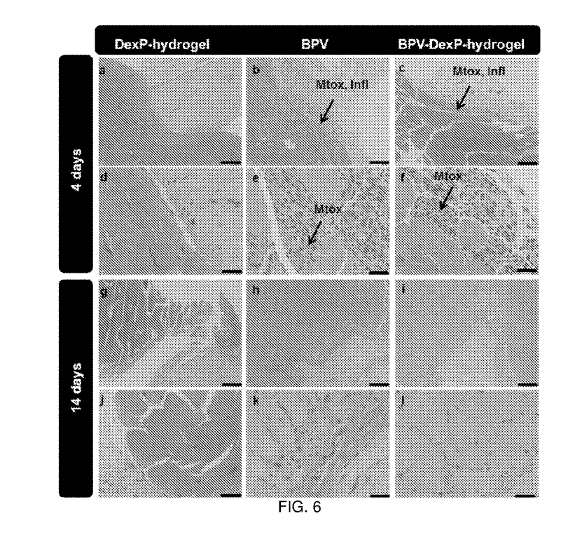

4 or 14 days after injection (n=6 at each time point), the sciatic nerve and surrounding tissues were harvested and processed for histology by hematoxylin-eosin staining (FIG. 6, Table 2). Overall, tissue reaction to all formulations was relatively benign. On day 4, animals injected with the DexP-hydrogel alone showed very mild muscle injury: a small number of muscle fibers with nuclear centralization. Animals injected with free bupivacaine solution had an inflammatory infiltrate consisting primarily of macrophages with lymphocytes and occasional neutrophils in the soft tissue surrounding the muscle. There was evidence of myotoxicity from the free bupivacaine solution, reflected by degenerating and regenerating myocytes predominantly in the perifascicular region of the muscle bundle. Tissue reaction to the BPV-DexP-hydrogel (Table 2) was similar to that to free bupivacaine, but with less inflammation. At day 14, tissue reaction to both free bupivacaine solution and BPV-DexP-hydrogel were diminished compared to reaction at day 4, almost back to normal.

FIG. 5 shows durations of motor and sensory blockade for the test materials. Data are mean+/−SD, n=4 for bupivacaine (BPV), mixture of bupivacaine and DexP (BPV+DexP) and the DexP-hydrogel groups, n=6 for BPV-DexP-hydrogel group. The concentration of bupivacaine was 5.0 mg/mL, the concentration of DexP was 25 mM, the molar ratio of DexP to Ca²⁺ was 1:1. Points falling below the diagonal line bisecting the graph (the line of unity) represent a relative sensory predominance in duration of nerve blockade, while those falling above have motor predominance.

FIG. 6 shows representative light microscopy of hematoxylin/eosin-stained (H&E) sections of muscle adjacent to the injection site, 4 and 14 days after injection of test materials: the DexP-hydrogel, bupivacaine solution (BPV), and the BPV-DexP-hydrogel. The concentration of bupivacaine was 5.0 mg/mL, the concentration of DexP was 25 mM, the molar ratio of DexP to Ca²⁺ was 1:1. Scale bars represent 200 micrometers (a-c, g-i) or 30 μm (d-f, j-l). “Mtox” and “Infl” represent myotoxicity and inflammation, respectively.

TABLE 2
Myotoxicity and inflammation scores.

| | 4 days | | 14 days | |
|---|---|---|---|---|
| | Myotoxicity | Inflammation | Myotoxicity | Inflammation |
| | (range 0-6) | (range 0-4) | (range 0-6) | (range 0-4) |
| Bupivacaine | 3.0 (3.0-3.75) | 2.0 (1.0-2.5) | 1.0 (0.5-1.0) | 1.0 (0.5-1.0) |
| solution | | | | |
| BPV-DexP- | 2.0 (1.0-3.5) | 1.0 (1.0-1.0) | 0.5 (0-1.0) | 0.5 (0-1.0) |
| hydrogel | | | | |
| p-value | p = 0.14 | p = 0.047 | p = 0.27 | p = 0.27 |

Data are median values with 25^{th} and 75^{th} percentiles, analyzed by the Mann-Whitney U-test; n = 4 for bupivacaine solution group, n = 6 for BPV-DexP-hydrogel group. The concentration of bupivacaine was 5.0 mg/mL, the concentration of DexP was 25 mM, the molar ratio of DexP and Ca²⁺ was 1:1.

In conclusion, these examples show shear-thinning and self-healing steroid drug-based hydrogels which were formed by supramolecular self-assembly and coordination interactions. These hydrogels exhibited rapid and complete recovery of mechanical properties within seconds following stress-induced flow. The release of steroid drugs could be controlled by tuning the molar ratio of drug and Ca²⁺. Subcutaneously injected hydrogels released steroid drug in vivo for ˜8 days, with no visible residue when release was complete. The DexP-hydrogel demonstrated glucocorticoid effects in vivo, minimizing the inflammation induced by bupivacaine and prolonging the duration of nerve blockade. The combination of high drug loading, easy injectability, and sustained release of steroids suggest potential application in a range of steroid-responsive diseases. The pharmacokinetics suggest potential applications as relatively short-term (˜1 week) depot systems for systemic effects.

### EXAMPLE 8

Following are additional materials and methods used in the above examples.

Materials and equipment. All the chemicals for hydrogel preparation were purchased from Sigma-Aldrich, Louis, Mo. TEM was performed on a Tecnai G² Spirit BioTWIN (Hillsboro, Oreg.), HR-TEM was performed on a JEOL 2010F (Tokyo, Japan). FTIR spectra were recorded by a Bruker TENSOR-27 FTIR spectrophotometer (Billerica, Mass.).

Preparation of hydrogel. Hydrogel were formed immediately by addition of different molar concentrations of CaCl₂ to solutions of dexamethasone phosphate disodium (DexP), betamethasone phosphate disodium (BetP), or hydrocortisone phosphate disodium (HydP). To form the DexP-hydrogel containing bupivacaine chlorhydrate (BPV), a DexP aqueous solution (100 mM) was added to a mixture of bupivacaine (10 mg/mL; approx. 30.8 mM) and Ca²⁺ (100 mM) in a volume ratio of 1:2:1.

ICP-MS and HPLC measurement. The DexP hydrogels (200 microliters) ([DexP]:[Ca²⁺]=2:1, 1:1, 2:3, [DexP]=25 mM) were washed with water (800 microliters), shaken and centrifuged to remove free Ca²⁺ or DexP and the nanofibers were collected. To determine the concentration of Ca²⁺, a sample of collected nanofibers was dissolved in 2% HNO₃ and analyzed by ICP-MS (Perkin Elmer Elan 6100, Perkin-Elmer Corp, Foster City, Calif.). To determine the concentration of DexP, another sample was incubated in phosphate buffered saline (PBS) (1 mL) in 1.5 mL centrifuge tubes, with changes of PBS every 12 hours. (PBS has approx. 10 mM phosphate, which can react with the Ca²⁺ in the hydrogels and hasten degradation.) This sample was dissolved in PBS completely after ˜2 days. The DexP content was determined by HPLC (Agilent Technologies, Santa Clara, Calif.).

Gel to sol transition temperature test. The gel to sol transition temperature was measured by the following method: a sealed glass vial containing the gel was immersed upside-down in a silicon oil bath. The temperature of the bath was raised at a rate of approximately 2° C. min⁻¹. The gel to sol transition temperature was defined as the temperature at which the gel began to collapse.

Rheology test. Rheological characterization was performed by a TA Instruments Discovery hybrid HR-2 rheometer (New Castle, Del.). Strain sweep and time sweep measurements were conducted at a frequency of 10.0 rad s⁻¹. All measurements were performed using a 20 mm plate geometry.

In vitro drug release. Hydrogel samples (200 microliters) or DexP aqueous solution (200 microliters, 25 mM) were placed into the upper chambers of 24-well Transwell plates (Costar 3470, Corning Incorporated, Corning, N.Y.) with a 0.4 micrometer pore size. 1 mL saline or PBS was placed in the lower chambers and incubated at 37° C. The saline or PBS was changed at predetermined time points, and the drug content was determined by HPLC (Agilent Technologies, Santa Clara, Calif.).

In vitro degradation. Hydrogel samples (200 microliters) were placed into glass vial. 1 mL saline or PBS was placed in the vials gently. At predetermined time points, the saline or PBS solution was removed gently. The hydrogel left in the vial were freeze-dried and weighed.

Cell viability. In vitro cell viability in the presence of DexP hydrogels was investigated in C2C12 cells (American Type Culture Collection, Manassas, Va.). In brief, C2C12 cells were grown and maintained in DMEM with 20% FBS and 1% Penicillin Streptomycin (Invitrogen, Waltham, Mass.) at 37° C. in 5% CO₂. Cells were seeded into the lower chamber of 24-well Transwell® plates (Costar 3470, pore size 0.4 micrometers, Corning Incorporated, Corning, N.Y.) at 50,000 cells/mL in DMEM with 2% horse serum and 1% penicillin streptomycin and incubated for 14 days to allow differentiation into myotubes. During differentiation, media were exchanged every 2-3 days. Then 200 microliters DexP hydrogel was added to the upper chamber and the cells were incubated with the gels for 1 day. (The hydrogels did not have direct contact with the cells.) The MTS were performed on one day after adding hydrogels.

Animal studies. Animal studies were conducted in compliance with protocols approved by the Children's Hospital Animal Care and Use Committee, and conformed to the guidelines of the International Association for the Study of Pain. Adult male Sprague-Dawley rats (Charles River Laboratories) weighing 320-400 g were housed in groups under a 6 AM to 6 PM light-dark cycle.

Pharmacokinetic of subcutaneous administration. Rats were injected subcutaneously with DexP-hydrogel (600 microliters, [DexP]=25 mM, [Ca²⁺]=25 mM; DexP]:[Ca²⁺]=1:1) or DexP PBS solution (200 microliters, [DexP]=25 mM), n=4 in each group. Blood samples (500 microliters) were taken at 30 min, 2 h, 4 h, 8 h, 1 day, 2 days, and 4 days. In animals treated with DexP-hydrogel only, blood was sampled at 5 and 8 days after injection. Immediately after sampling, plasma was separated from the blood by centrifugation (4400 RPM, 15 min, 4° C.) and frozen at ˜20° C. until analyzed. An equal volume of acetonitrile was added to the plasma, which was then stored at 4° C. for 2 days, then centrifuged (14000 RPM, 15 min, 4° C.) to remove protein precipitates. The clear supernatant was analyzed by HPLC (Agilent Technologies, Santa Clara, Calif.) for dexamethasone content. Data for dexamethasone content are reported as the integration of the dexamethasone and dexamethasone phosphate peaks on HPLC spectra, since dexamethasone phosphate is converted to dexamethasone in plasma. Dexamethasone phosphate standards ranging in concentration from 0.5 to 87.5 micrograms/mL were prepared in plasma obtained from untreated rats, to use in the standard curve for determining dexamethasone concentrations in measured samples.

Sciatic nerve blockade. Under brief isoflurane-oxygen anesthesia, the animals were injected with 0.3 mL of test material using a 23 G needle, which was introduced postero-medial to the greater trochanter until contact with bone.

Sensory nerve block was assessed by a modified hotplate test, as reported previously. The plantar surface of the rat's hind paw was placed on a pre-heated hot plate at 56° C. The time (termed thermal latency) that the animal would leave its hindpaw on the hotplate was recorded. Longer latencies reflected deeper degrees of local anesthesia. Pristine animals have latencies of about 2 s. After 12 s, the hindpaw was removed from the hotplate by the investigator to avoid tissue thermal injury or the development of hyperalgesia. Measurements were repeated 3 times at each timepoint. The duration of block was calculated as the time required for thermal latency to return to a value of 7 s from a higher value; 7 s is the midpoint between a baseline thermal latency of 2 seconds in adult rats, and a maximal latency of 12 s.

Motor blockade was assessed by a weight-bearing test. In brief, the animal was held over a digital balance and bore weight with one hindpaw at a time. The maximum weight the animal could bear was recorded. The duration of motor blockade was calculated as the time for weight bearing to return halfway to normal from maximal block. The halfway point for each rat was defined as [(highest weight borne by either leg)−(lowest weight borne by blocked leg)]/2+(lowest weight borne by blocked leg).

Histology. Rats were euthanized by carbon dioxide 4 days or 14 days after injection. The nerve and surrounding tissue were harvested and underwent standard procedures to produce hematoxylin and eosin-stained slides. The samples were scored for inflammation (0-4) and myotoxicity (0-6). The inflammation score was a subjective quantification of severity, where 0 was normal and 4 was severe inflammation. The myotoxicity score was determined by the nuclear internalization and regeneration of myocytes, two representative characteristics of local anesthetics myotoxicity. Nuclear internalization was characterized by myocytes having nuclei located away from its usual location at the periphery of the cell. Regeneration was characterized by the presence of shrunken myocytes with basophilic cytoplasm. The scoring scale was as follows: 0=normal; 1=per fascicular internalization; 2=deep internalization (>5 cell layers); 3=perifascicular regeneration; 4=deep tissue regeneration (>5 cell layers); 5=hemefascicular regeneration; 6=holofascicular regeneration.

Statistical Analysis. Drug release, cytotoxicity and duration of nerve block data were reported as mean+/−standard deviations (SD), and analyzed by t-tests. Histology scores were reported as medians with 25^{th} and 75^{th} percentiles, and analyzed by the Mann-Whitney U-test. Statistical significance was determined when p<0.05.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A composition, comprising:
a steroid comprising a phosphate moiety and a core portion, the core portion having a logP value greater than or equal to 1, complexed via Ca²⁺ and/or Ba²⁺ metal ions to form a hydrogel, wherein the steroid forms at least 50% by dry weight of the hydrogel.

2. The composition of claim 1, wherein the phosphate moiety is bonded to a carboxylate within the steroid.

3. The composition of claim 2, wherein the carboxylate is bonded to a D-ring of the steroid.

4. The composition of any one of claims 1-3, wherein the core portion of the steroid comprises a gonane structure.

5. The composition of any one of claims 1-4, wherein the core portion of the steroid comprises a methyl group at the 10 position, a methyl group at the 13 position, and/or a carboxylate moiety at the 17 position.

6. The composition of claim 5, wherein the core portion of the steroid further comprises an oxo moiety at the 3 position.

7. The composition of any one of claim 5 or 6, wherein the core portion of the steroid further comprises a hydroxyl moiety at the 17 position.

8. The composition of any one of claims 1-7, wherein the steroid comprises a carboxylate at the 17 position.

9. The composition of any one of claims 5-8, wherein the core portion of the steroid comprises an acetyl moiety at the 17 position.

10. The composition of claim 9, wherein the phosphate moiety is bonded to the methyl group of the acetyl moiety.

11. The composition of any one of claims 1-9, wherein the phosphate moiety is attached at the 17 position of the core portion of the steroid.

12. The composition of any one of claims 1-11, wherein the core portion of the steroid does not comprise a carboxylic acid group.

13. The composition of any one of claims 1-12, wherein the core portion of the steroid comprises at least one hydroxyl group.

14. The composition of any one of claims 1-13, wherein each face of the steroid comprises an equal number of hydroxyl groups.

15. The composition of any one of claims 1-14, wherein the steroid comprises a corticosteroid.

16. The composition of any one of claims 1-15, wherein the steroid comprises a glucocorticoid.

17. The composition of any one of claims 1-16, wherein the steroid has the following structure:

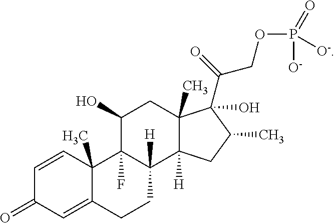

18. The composition of any one of claims 1-16, wherein the steroid has the following structure:

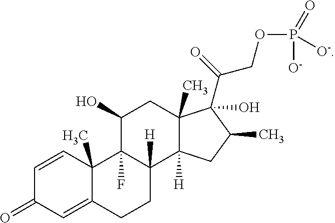

19. The composition of any one of claims 1-16, wherein the steroid has the following structure:

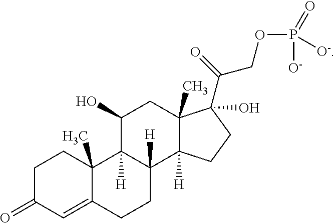

20. The composition of any one of claims 1-19, wherein the steroid forms at least 75% by dry weight of the hydrogel.

21. The composition of any one of claims 1-20, wherein the core portion of the steroid has a logP value greater than or equal to 1.2.

22. The composition of any one of claims 1-21, wherein the core portion of the steroid has no formal charged moieties at a pH of 7.

23. The composition of any one of claims 1-22, wherein the steroid is present within the hydrogel at a concentration of at least 2.5 mM.

24. The composition of any one of claims 1-23, wherein the steroid is present within the hydrogel at a concentration of at least 8 mM.

25. The composition of any one of claims 1-24, wherein the steroid is present within the hydrogel at a concentration of at least 10 mM.

26. The composition of any one of claims 1-25, wherein the concentration of steroid within the hydrogel is at most 25 mM.

27. The composition of any one of claims 1-26, wherein the metal ions are present within the hydrogel at a concentration of at least 2.5 mM.

28. The composition of any one of claims 1-27, wherein the metal ions are present within the hydrogel at a concentration of at least 8 mM.

29. The composition of any one of claims 1-28, wherein the metal ions are present within the hydrogel at a concentration of at least 10 mM.

30. The composition of any one of claims 1-29, wherein the steroid and the metal ion are present at a molar ratio of between about 2:1 and about 1:2.

31. The composition of any one of claims 1-30, wherein the metal ion comprises Ca²⁺.

32. The composition of any one of claims 1-31, wherein at least 50 mol % of the metal ions are Ca²⁺.

33. The composition of any one of claims 1-32, wherein the metal ion comprises Ba²⁺.

34. The composition of any one of claims 1-33, wherein at least 50 mol % of the metal ions are Ba²⁺.

35. The composition of any one of claims 1-34, wherein the hydrogel further comprises a salt.

36. The composition of any one of claims 1-35, wherein the hydrogel further comprises an anesthetic.

37. The composition of claim 36, wherein the anesthetic comprises bupivacaine.

38. The composition of any one of claims 1-37, wherein the hydrogel comprises nanofibers.

39. The composition of any one of claims 1-38, wherein the hydrogel is shear-thinning.

40. The composition of any one of claims 1-39, wherein the hydrogel is exhibits decreased viscosity when subjected to shear strain.

41. The composition of any one of claims 1-40, wherein the hydrogel is self-healing.

42. The composition of any one of claims 1-41, wherein the hydrogel is thixotropic.

43. A needle, comprising the composition of any one of claims 1-42.

44. A syringe, comprising the composition of any one of claims 1-42.

45. An implant, comprising the composition of any one of claims 1-42.

46. A composition, comprising:
a steroid comprising a phosphate moiety and a core portion, the core portion having no formal charged moieties at a pH of 7, the steroid complexed via Ca²⁺ and/or Ba²⁺ metal ions to form a nanofiber.

47. The composition of claim 46, wherein the nanofiber has an average cross-sectional diameter between 5 nm and 25 nm.

48. The composition of any one of claim 46 or 47, wherein the nanofiber has an average cross-sectional diameter between 5 nm and 15 nm.

49. The composition of any one of claims 46-48, wherein the phosphate moiety is bonded to a carboxylate within the steroid.

50. The composition of claim 49, wherein the carboxylate is bonded to a D-ring of the steroid.

51. The composition of any one of claims 46-50, wherein the core portion of the steroid comprises a gonane structure.

52. The composition of any one of claims 46-51, wherein the core portion of the steroid comprises a methyl group at the 10 position, a methyl group at the 13 position, and/or a carboxylate moiety at the 17 position.

53. The composition of claim 52, wherein the core portion of the steroid further comprises an oxo moiety at the 3 position.

54. The composition of any one of claim 52 or 53, wherein the core portion of the steroid further comprises a hydroxyl moiety at the 17 position.

55. The composition of any one of claims 46-54, wherein the steroid comprises a carboxylate at the 17 position.

56. The composition of any one of claims 52-55, wherein the core portion of the steroid comprises an acetyl moiety at the 17 position.

57. The composition of claim 56, wherein the phosphate moiety is bonded to the methyl group of the acetyl moiety.

58. The composition of any one of claims 46-56, wherein the phosphate moiety is attached at the 17 position of the core portion of the steroid.

59. The composition of any one of claims 46-58, wherein the core portion of the steroid does not comprise a carboxylic acid group.

60. The composition of any one of claims 46-59, wherein the core portion of the steroid comprises at least one hydroxyl group.

61. The composition of any one of claims 46-60, wherein each face of the steroid comprises an equal number of hydroxyl groups.

62. The composition of any one of claims 46-61, wherein the steroid comprises a corticosteroid.

63. The composition of any one of claims 46-62, wherein the steroid comprises a glucocorticoid.

64. The composition of any one of claims 46-63, wherein the steroid has the following structure:

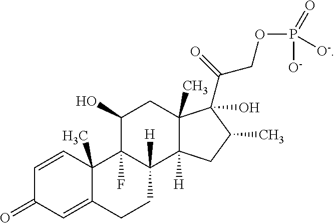

65. The composition of any one of claims 46-63, wherein the steroid has the following structure:

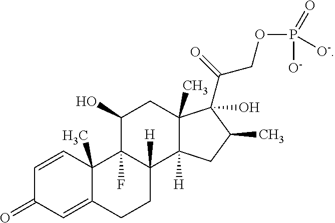

66. The composition of any one of claims 46-63, wherein the steroid has the following structure:

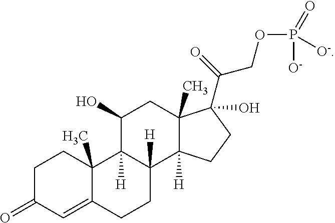

67. The composition of any one of claims 46-66, wherein the steroid forms at least 75% by dry weight of the nanofiber.

68. The composition of any one of claims 46-67, wherein the core portion of the steroid has a logP value greater than or equal to 1.

69. The composition of any one of claims 46-68, wherein the core portion of the steroid has a logP value greater than or equal to 1.2.

70. The composition of any one of claims 46-69, wherein the steroid and the metal ion are present at a molar ratio of between about 2:1 and about 1:2.

71. The composition of any one of claims 46-70, wherein the metal ion comprises Ca2+.

72. The composition of any one of claims 46-71, wherein at least 50 mol % of the metal ions are Ca²⁺.

73. The composition of any one of claims 46-72, wherein the metal ion comprises Ba^{2±}.

74. The composition of any one of claims 46-73, wherein at least 50 mol % of the metal ions are Ba²⁺.

75. The composition of any one of claims 46-74, wherein the nanofiber is a component of a hydrogel.

76. The composition of claim 75, wherein the steroid is present within the hydrogel at a concentration of at least 2.5 mM.

77. The composition of any one of claim 75 or 76, wherein the steroid is present within the hydrogel at a concentration of at least 8 mM.

78. The composition of any one of claims 75-76, wherein the steroid is present within the hydrogel at a concentration of at least 10 mM.

79. The composition of any one of claims 75-78, wherein the concentration of steroid within the hydrogel is at most 25 mM.

80. The composition of any one of claims 75-79, wherein the metal ions are present within the hydrogel at a concentration of at least 2.5 mM.

81. The composition of any one of claims 75-80, wherein the metal ions are present within the hydrogel at a concentration of at least 8 mM.

82. The composition of any one of claims 75-81, wherein the metal ions are present within the hydrogel at a concentration of at least 10 mM.

83. The composition of any one of claims 75-82, wherein the hydrogel further comprises a salt.

84. The composition of any one of claims 75-83, wherein the hydrogel further comprises an anesthetic.

85. The composition of claim 84, wherein the anesthetic comprises bupivacaine.

86. The composition of any one of claims 75-85, wherein the hydrogel comprises nanofibers.

87. The composition of any one of claims 75-86, wherein the hydrogel is shear-thinning.

88. The composition of any one of claims 75-87, wherein the hydrogel exhibits decreased viscosity when subjected to shear strain.

89. The composition of any one of claims 75-88, wherein the hydrogel is self-healing.

90. The composition of any one of claims 75-89, wherein the hydrogel is thixotropic.

91. A needle, comprising the composition of any one of claims 75-90.

92. A syringe, comprising the composition of any one of claims 75-90.

93. An implant, comprising the composition of any one of claims 75-90.

94. A composition, comprising:
a hydrogel comprising a steroid and Ca²⁺ and/or Ba²⁺ metal ions, the steroid comprising a phosphate moiety and being present within the hydrogel at a concentration of at least 8 mM, and the metal ions being present within the hydrogel at a concentration of at least 8 mM.

95. A composition, comprising:
a nanofiber formed from a complex of (a) a steroid comprising a phosphate moiety, and (b) Ca²⁺ and/or Ba²⁺ metal ions, the steroid and the metal ions present within the nanofiber at a molar ratio of between about 2:1 and about 1:2.
